(19) United States
(12) Patent Application Publication
(10) Pub. No.: US 2019/0307719 A1
(43) Pub. Date: Oct. 10, 2019

(54) CANNABIS COMPOSITION

(71) Applicant: ZELDA THERAPEUTICS OPERATIONS PTY LTD, Perth (AU)

(72) Inventors: Harry KARELIS, Perth (AU); Mara GORDON, Bodega Bay, CA (US); Stewart SMITH, Bodega Bay, CA (US); Stewart WASHER, Stirling (AU)

(21) Appl. No.: 16/322,898

(22) PCT Filed: Aug. 3, 2017
(86) PCT No.: PCT/AU2017/050815

§ 371 Date: Feb. 1, 2019

Related U.S. Application Data
(60) Provisional application No. 62/370,303, filed on Aug. 3, 2016.

(51) Int. Cl. A61K 31/352; A61K 31/05; A61K 9/00; A61K 31/045

(52) U.S. Cl. CPC A61K 31/352; A61K 31/045; A61K 9/0014; A61K 31/05

(57) ABSTRACT

The invention relates to a method for treating a skin disorder. In particular, the invention provides a method for treating a skin disorder, comprising administering to a patient in need thereof an effective amount of the pharmaceutical composition comprising a Cannabis extract and optionally one or more pharmaceutically acceptable carriers, diluents, adjuvants, excipients or any combination thereof, the Cannabis extract comprising at least 75% by weight of a main cannabinoid.

### FIELD

The invention relates to a method for treating a skin disorder. The invention also relates to a topical pharmaceutical composition comprising an extract from a Cannabis plant, and its use in the treatment of the skin disorder.

### BACKGROUND

The biological activity of Cannabis is well known, and has led it to become a “recreational” drug. However, with the discovery of a class of cannabinoid (CB) receptors, and the relaxation of laws regulating Cannabis use—in some jurisdictions decriminalisation—there now exists the opportunity to explore the potential of Cannabis as a source of new therapeutics.

There is also a growing number of patients suffering from diseases, such as skin disorders, that are seeking natural remedies as alternative or complementary therapy.

Accordingly, there is a continuing need to develop new treatments for skin disorders, which are derived, at least in part, from a natural source.

### SUMMARY

The invention provides a method of treating a skin disorder comprising topically administering to a patient in need thereof an effective amount of a pharmaceutical composition comprising a Cannabis extract.

In one aspect, there is provided a topical pharmaceutical composition comprising an effective amount of a Cannabis extract and a topical delivery system.

In another aspect, there is provided a pharmaceutical composition comprising a Cannabis extract and optionally one or more pharmaceutically acceptable carriers, diluents, adjuvants, excipients or any combination thereof, wherein the extract comprises at least 75% by weight of a main (or primary) cannabinoid.

In one embodiment, the pharmaceutical composition comprises Δ⁹-Tetrahydrocannabinol (THC) or cannabidiol (CBD). In further embodiments the Cannabis extract further comprises one or more secondary cannabinoids selected from Cannabinodiol (CBN), Cannabichromanone (CBC), Δ⁹-Tetrahydrocannabinolic acid (THCA) and Cannabigerol (CBG).

In one embodiment, the pharmaceutical composition of the present invention further comprises one or more terpenes selected from the group consisting of beta-myrcene, linalool, nerolidol, limonene, alpha-bisabolol, camphene, delta-s-carene, beta-caryophyllene, caryophyllene oxide, p-cymene, geraniol, humulene, ocimene, pinene, and alpha-terpinene.

In one aspect the pharmaceutical composition comprises limonene in an amount of at least about 5.4% by weight of the terpene fraction.

In some embodiments, the present invention provides a topical pharmaceutical composition comprising an effective amount of a Cannabis extract and a topical delivery system.

The topical pharmaceutical composition comprises a topical delivery system that comprises two or more of Bergamot essential oil, Cedarwood essential oil, Chamomile essential oil, Clary sage essential oil, Cypress essential oil, Eucalyptus essential oil, Fennel essential oil, Frankincense essential oil, Geranium essential oil, Hyssop essential oil, Jasmine essential oil, Juniper essential oil, Lavender essential oil, Lemon essential oil, Lemongrass essential oil, Marjoram essential oil, Melaleuca essential oil, Myrrh essential oil, Myrtle essential oil, Neem essential oil, Orange essential oil, Oregano essential oil, Palma rosa essential oil, Patchouli essential oil, Peppermint essential oil, Rose essential oil, Rosemary essential oil, Rosewood essential oil, Sage essential oil, Sandalwood essential oil, Tangerine essential oil, Tea tree essential oil, Thyme essential oil, Ylang ylang essential oil, Sesame oil, Olive oil, Arnica essential oil, Lavender Spike essential oil, Cinnamon Leaf essential oil, Rosemary Cineole essential oil, Coconut oil, Bees wax and Hemp oil.

Preferably, the topical pharmaceutical composition comprises a topical delivery system that comprises two or more of Sesame oil, Olive oil, Arnica essential oil, Lavender essential oil, Lavender Spike essential oil, Frankincense essential oil, Lemongrass essential oil, Cinnamon Leaf essential oil, Rosemary Cineole essential oil, Rosemary essential oil, Bergamot essential oil, Myrrh essential oil, Sage essential oil, Coconut oil, Bees wax and Hemp oil.

In a further aspect, there is provided use of the Cannabis extract in the preparation of a medicament for treating a skin disorder.

In yet another aspect, there is provided the pharmaceutical composition or topical pharmaceutical composition for treating a skin disorder.

### DESCRIPTION OF EMBODIMENT(S)

The present invention provides a pharmaceutical composition. The pharmaceutical composition comprises a Cannabis extract. The pharmaceutical composition is a topical pharmaceutical composition, meaning that it is suitable for administering the active components of the Cannabis extract topically. The topical administration is typically local administration; however, in some embodiments, the topical administration may be systemic. The topical administration may preferably be administration directly to the skin of a patient.

The inventors have found that topical administration of a Cannabis extract is useful for treating a range of skin disorders.

### Topical Delivery System

The topical pharmaceutical composition comprises a topical delivery system. The topical delivery system may advantageously enhance the delivery of the active components of the Cannabis extract to the skin of the patient.

The topical delivery system preferably comprises two or more pharmaceutically acceptable components. By “pharmaceutically acceptable”, it is meant that the components are compatible with the other ingredients of the composition and are not deleterious to a subject upon or following administration. It is believed that the topical delivery system may enhance the permeability of the patient's skin to increase the local absorption of the active components of the Cannabis extract. The topical delivery system may comprise three, four, five, six, seven, eight, nine, ten, eleven, twelve or more components.

The pharmaceutically acceptable components of the topical delivery system may be selected from an essential oil (e.g. an oil derived from a plant, such as a herb), a wax, or a combination thereof. The pharmaceutically acceptable components of the topical delivery system may be selected from: Bergamot essential oil, Cedarwood essential oil, Chamomile essential oil, Clary sage essential oil, Cypress essential oil, Eucalyptus essential oil, Fennel essential oil, Frankincense essential oil, Geranium essential oil, Hyssop essential oil, Jasmine essential oil, Juniper essential oil, Lavender essential oil, Lemon essential oil, Lemongrass essential oil, Marjoram essential oil, Melaleuca essential oil, Myrrh essential oil, Myrtle essential oil, Neem essential oil, Orange essential oil, Oregano essential oil, Palma rosa essential oil, Patchouli essential oil, Peppermint essential oil, Rose essential oil, Rosemary essential oil, Rosewood essential oil, Sage essential oil, Sandalwood essential oil, Tangerine essential oil, Tea tree essential oil, Thyme essential oil, Ylang ylang essential oil, Sesame oil, Olive oil, Arnica essential oil, Lavender Spike essential oil, Cinnamon Leaf essential oil, Rosemary Cineole essential oil, Coconut oil, Bees wax and Hemp oil. Each pharmaceutically acceptable component may be present in the same or different amount. For example, the topical delivery system may comprise each pharmaceutically acceptable component in an amount from 0% to 95% by weight.

Individual ingredients of the topical delivery system may also comprise active compounds useful for the treatment of the skin disorder. For example, essential oils that may be useful for the treatment of a skin disorder include: Bergamot essential oil, Cedarwood essential oil, Chamomile essential oil, Clary sage essential oil, Cypress essential oil, Eucalyptus essential oil, Fennel essential oil, Frankincense essential oil, Geranium essential oil, Hyssop essential oil, Jasmine essential oil, Juniper essential oil, Lavender essential oil, Lemon essential oil, Lemongrass essential oil, Marjoram essential oil, Melaleuca essential oil, Myrrh essential oil, Myrtle essential oil, Neem essential oil, Orange essential oil, Oregano essential oil, Palma rosa essential oil, Patchouli essential oil, Peppermint essential oil, Rose essential oil, Rosemary essential oil, Rosewood essential oil, Sage essential oil, Sandalwood essential oil, Tangerine essential oil, Tea tree essential oil, Thyme essential oil, and Ylang ylang essential oil, or a combination thereof.

One exemplary topical pharmaceutical composition comprising a topical delivery system is outlined in Table 1 below.

TABLE 1
Topical Pharmaceutical Composition

| Ingredient | Amount (wt %) |
|---|---|
| Arnica essential oil | 0-10 (e.g. 0.001-5) |
| Lavender essential oil | 0-10 (e.g. 0.001-5) |
| Lavender Spike essential oil | 0-10 (e.g. 0.001-5) |
| Frankincense essential oil | 0-10 (e.g. 0.001-5) |
| Lemongrass essential oil | 0-10 (e.g. 0.001-5) |
| Cinnamon Leaf essential oil | 0-10 (e.g. 0.001-5) |
| Rosemary Cineole essential oil | 0-10 (e.g. 0.001-5) |
| Rosemary essential oil | 0-10 (e.g. 0.001-5) |
| Bergamot essential oil | 0-10 (e.g. 0.001-5) |
| Myrrh essential oil | 0-10 (e.g. 0.001-5) |
| Sage essential oil | 0-10 (e.g. 0.001-5) |
| Coconut oil | 0-95 (e.g. 50-95 or 70-90) |
| Bees wax | 0-30 (e.g. 5-25) |
| Cannabis extract | 0-20 (e.g. 0.001-10 or 0.01-5) |

### Cannabis Extract

Cannabis plants produce a diverse array of secondary metabolites, including cannabinoids, terpenes and terpenoids, sterols, triglycerides, alkanes, squalenes, tocopherols, carotenoids and alkaloids. The mix of these secondary metabolites varies depending on several factors, including Cannabis variety, part of the Cannabis plant extracted, method of extraction, processing of the extract, and season.

There are several varieties of Cannabis plant, which have been described under two distinct naming conventions. One of these conventions identifies three distinct species of Cannabis plant, namely Cannabis sativa Linnaeus, Cannabis indica LAM., and Cannabis ruderalis. Another convention identifies all Cannabis plants as belonging to the Cannabis sativa L. species, with the various varieties divided amongst several subspecies, including: Cannabis sativa ssp. sativa and ssp. indica. As used herein, the term “Cannabis” refers to any and all of these plant varieties.

Extracts of Cannabis may be prepared by any means known in the art. The extracts may be formed from any part of the Cannabis plant containing cannabinoid, terpene and terpenoid compounds. Extracts may be formed by contacting an extractant with a leaf, seed, trichome, flower, keif, shake, bud, stem or a combination thereof. In some embodiments, the extract is formed from the flowers and shake of a Cannabis plant. Any suitable extractant known in the art may be used, including, for example, alcohols (e.g. methanol, ethanol, propanol, butanol, propylene glycol etc.), water, hydrocarbons (e.g. butane, hexane, etc.), oils (e.g. olive oil, vegetable oil, essential oil, etc.), a solvent (e.g. ethyl acetate, polyethylene glycol, etc.) or a supercritical fluid (e.g. liquid CO₂). The extractant may be completely or partially removed prior to incorporation of the Cannabis extract into the pharmaceutical composition, or it may be included in the pharmaceutical composition as a carrier. The extractant may be removed by heating the extract optionally under reduced pressure. It will be appreciated that some of the more volatile plant metabolites (such as terpenes) may also be removed with the extractant. Accordingly, in some embodiments, removing the extractant may enrich the cannabinoid fraction of the extract. In some embodiments, the extract is filtered to remove particulate material, for example, by passing the extract through filter paper or a fine sieve (e.g. a sieve with pore sizes of 5 μm).

In some embodiments, the Cannabis extract is formed by applying heat and pressure to the plant material. Typically, in these embodiments, no extractant is required.

In some embodiments, the Cannabis extract is a Cannabis oil. As used herein, a “Cannabis oil” is an extract formed by contacting at least a part of a Cannabis plant with an oil. The extracting oil may optionally be removed. Extracting oils may be selected from olive oil, hemp oil, sesame oil, coconut oil, vegetable oil, canola oil, grape seed oil, almond oil, medium-chain triglyceride (MCT) oil, and any other edible oil, or a combination thereof.

The term “cannabinoid” as used herein relates to any cannabinoid that have been isolated from a Cannabis plant or synthetically created to have activity involving the endocannabinoid system.

The term “cannabinoid fraction” is used to describe the combination of cannabinoid compounds present in the Cannabis extract.

The term “terpenes” or “terpenoids” as used herein refers to a class of hydrocarbon molecules, which often provide a unique smell. Terpenes are derived from units of isoprene, which has the molecular formula C₅H₈. The basic molecular formula of terpenes are multiples of the isoprene unit, i.e. (C₅H₈)ₙ, where n is the number of linked isoprene units. Terpenoids are terpene compounds that have been further metabolised in the plant, typically through an oxidative process, and therefore usually contain at least one oxygen atom.

The term “terpene fraction” is used to describe the combination of terpene and terpenoid compounds present in the Cannabis extract.

Cannabis Extract

The Cannabis extract comprises a cannabinoid fraction and a terpene fraction.

In some embodiments, the Cannabis extract contains high amounts (e.g. greater than 75% by weight) of the cannabinoid fraction. In some embodiments, the Cannabis extract may comprise the cannabinoid fraction in an amount of about 75% to about 99.999% by weight, for example, about 80% to about 99.999%, about 80% to about 99.99%, about 80% to about 99.9%, or about 80% to about 99.5% by weight of the Cannabis extract. In some embodiments, the Cannabis extract comprises about 0.001% to about 20% by weight of non-cannabinoids, for example, about 0.001% to about 15% by weight or about 0.001% to about 10% by weight non-cannabinoids.

In some embodiments, one or more additional compounds (e.g. cannabinoid, terpene or terpenoid compounds) may be added to the Cannabis extract. The addition of compounds may be to compensate for natural variations in the relative amounts of certain compounds being expressed in the Cannabis plant. The added compounds may be synthetic versions of the desired compounds, they may be purified compounds obtained from other Cannabis extracts, or they may be added by blending two or more extracts.

To date, over 100 cannabinoids have been identified in Cannabis plants. A comprehensive list of these cannabinoids may be found in Mahmoud A. El Sohly and Waseem Gul, “Constituents of Cannabis Sativa.” In Handbook of Cannabis Roger Pertwee (Ed.) Oxford University Press (2014) (ISBN: 9780199662685). Cannabinoids that have been identified in Cannabis plants include: Cannabigerol (E)-CBG-C5, Cannabigerol monomethyl ether (E)-CBGM-C5 A, Cannabigerolic acid A (Z)-CBGA-C5 A, Cannabigerovarin (E)-CBGV-C3, Cannabigerolic acid A (E)-CBGA-C5 A, Cannabigerolic acid A monomethyl ether (E)CBGAM-C5 A and Cannabigerovarinic acid A (E)-CBGVAC3A); (±)-Cannabichromene CBC-C5, (±)-Cannabichromenic acid A CBCA-C5 A, (±)-Cannabivarichromene, (±)-Cannabichromevarin CBCV-C3, (±)-Cannabichromevarinic acid A CBCVA-C3 A); (−)-Cannabidiol CBD-C5, Cannabidiol momomethyl ether CBDMC5, Cannabidiol-C4 CBD-C4, (−)-Cannabidivarin CBDVC3, Cannabidiorcol CBD-CI, Cannabidiolic acid CBDA-C5, Cannabidivarinic acid CBDVA-C3); Cannabinodiol CBNDC5, Cannabinodivarin CBND-C3); Δ⁹-Tetrahydrocannabinol C5, Δ⁹-Tetrahydrocannabinol-C4 Δ⁹-THCC4, Δ⁹-Tetrahydrocannabivarin Δ⁹-THCV-C3, Δ⁹-Tetrahydrocannabiorcol, Δ⁹-Tetrahydrocannabinolic acid Δ⁹-THCA-C5 A, Δ⁹-Tetrahydrocannabinolic acid B, Δ⁹-THCA-C5 B, Δ⁹-Tetrahydrocannabinolic acid-C4 A and/or B Δ⁹-THCA-C4 A and/or B, Δ⁹-Tetrahydro-cannabivarinic acid A Δ⁹-THCVA-C3 A, Δ⁹-Tetrahydrocannabiorcolic acid A and/or B Δ⁹-THCOA-CI A and/or B), (−)-Δ⁸-trans-(6aR,10aR)-Δ⁸-Tetrahydrocannabinol (−)-Δ⁸-trans-(6aR,10aR)-Tetrahydrocannabinolic acid A Δ⁸-THCA-C5 A, (−)-(6a5,10aR)-Δ⁹-Tetrahydrocannabinol (−)-cis-Δ⁹-THC-C5); Cannabinol CBN-C5, Cannabinol-C4 CBN-C4, Cannabivarin CBN-C3, Cannabinol C2 CBN-C2, Cannabiorcol CBN-CI, Cannabinolic acid A CBNA-C5 A, Cannabinol methyl ether CBNM-C5, (−)-(9R,10R)-trans-Cannabitriol (−)-trans-CBT-C5, (+)-(9S,10S)-Cannabitriol (+)-trans-CBT-C5, (±)-(9R,10S/9S,10R)-); Cannabitriol (±)-cis-CBT-C5, (−)-(9R,10R)-trans-10-O-Ethyl-cannabitriol (−)-trans-CBT-OEt-C5, (±)-(9R,10R/95,10S)-Cannabitriol-C3 (±)-trans-CBT-C3, 8,9-Dihydroxy-Δ6a(10a)-tetrahydrocannabinol 8,9-Di-OH-CBT-C5, Cannabidiolic acid A cannabitriol ester CBDA-C5 9-OH-CBT-C5 ester, (−)-(6aR,9S,10S,10aR)-9,10-Dihydroxyhexahydrocannabinol, Cannabiripsol, Cannabiripsol-C5, (−)-6a,7,10a-Trihydroxy-,6,⁹-tetrahydrocannabinol (−)-Cannabitetrol, 10-Oxo-Δ6a(10a)tetrahydrocannabinol OTHC); (5aS,6S,9R,9aR)-Cannabielsoin CBE-C5, (5aS,6S,9R,9aR)-C3-Cannabielsoin CBE-C3, (5aS,6S,9R,9aR)-Cannabielsoic acid A CBEA-C5 A, (5aS,6S,9R,9aR)-Cannabielsoic acid B CBEA-C5 B; (5aS,6S,9R,9aR)-C3-Cannabielsoic acid B CBEA-C3 B, Cannabiglendol-C3 OH-iso-HHCV-C3, Dehydrocannabifuran DCBF-C5, Cannabifuran CBF-C5), (−)-Δ⁷-trans-(1R,3R,6R)-Isotetrahydrocannabinol, (±)-Δ⁷-1,2-cis-(1R,3R,6S/1S,3S,6R)-Isotetrahydrocannabivarin, (−)-Δ⁷-trans-(1R,3R,6R)-Isotetrahydrocannabivarin; (±)-(IaS,3aR,8bR,8cR)-Cannabicyclol CBL-C5, (±)-(1aS,3aR,8bR,8cR)-Cannabicyclolic acid A CBLA-C5 A, (±)-(1aS,3aR,8bR,8cR)-Cannabicyclovarin CBLV-C3; Cannabicitran CBTC5; Cannabichromanone CBCN-C5, CannabichromanoneC3 CBCN-C3, and Cannabicoumaronone CBCON-C5.

The Cannabis extract may comprise at least 75% by weight of a main cannabinoid. The main cannabinoid may be Δ⁹-tetrahydrocannabinol (THC) or cannabidiol (CBD). The Cannabis extract may comprise the main cannabinoid in an amount of at least 76%, 77%, 78%, 79%, 80%, 81%, 82%, 83%, 84% or 85% by weight of the extract.

Typically, the Cannabis extract further comprises one or more secondary cannabinoids. The secondary cannabinoids may be selected from Cannabinodiol (CBN), Cannabichromanone (CBC), Δ⁹-Tetrahydrocannabinolic acid (THCA) and Cannabigerol (CBG). THC or CBD may also be present in the Cannabis extract as a secondary cannabinoid. Typically, each secondary cannabinoid is present in an amount from 0.001% to about 20% by weight of the extract, for example, about 0.001% to about 15% or about 0.01% to about 15% by weight of the extract.

In some embodiments, certain cannabinoids may be absent, or present in non-detectable amounts (e.g. less than 0.001% by weight of the analyte). In some embodiments, the Cannabis extract may exclude one or more of the following cannabinoids: Δ⁹-Tetrahydrocannabinolic acid (THCA), Cannabidiol (CBD), Δ⁹-Tetrahydrocannabivarin (THCV), Cannabidiolic acid (CBDA), Cannabigerolic acid (CBGA), Cannabinodiol (CBN) and Cannabichromanone (CBC).

The Cannabis extract comprises non-cannabinoid compounds, which typically includes a terpene fraction, i.e. terpenes and terpenoids. In some embodiments, the Cannabis extract comprises a terpene fraction in an amount of less than 20% by weight, for example, less than 15%, 10%, 9%, 8%, 7%, 6%, 5%, 4%, 3%, 2% or 1% by weight of the extract. In some embodiments, the Cannabis extract may comprise terpene and terpenoid compounds in an amount of more than 0.001% by weight of the extract, for example, more than 0.001%, 0.005%, 0.01%, 0.05%, 0.1%, 0.5%, or 1% of the total weight of the extract. In some embodiments, the Cannabis extract comprises about 0.001% to about 20% by weight of terpene and terpenoid compounds, for example, about 0.001% to about 15% by weight, about 0.001% to about 10% by weight, about 0.001% to about 6% by weight or about 0.001% to about 5% by weight of the composition.

Typically, the terpene fraction in the plant material used to form the extract may have a different terpene/terpenoid profile than the terpene profile of the final extract, both in terms of the amounts of specific compounds in the terpene fraction and the weight of the terpene fraction relative to the other components. For example, a Cannabis flower may comprise about 20% by weight cannabinoids and about 3% by weight terpenes. Following extraction and concentration (i.e. removal of the extractant), the amount of cannabinoids may increase to an amount of about 50-90% by weight and the terpene fraction may amount to about 0.1-6% by weight of the Cannabis extract. This typical scenario shows that while the cannabinoids are concentrated when the extractant is removed, the relative amount of the terpene fraction is reduced, likely due to the volatility of many of the terpenes/terpenoids present in the terpene fraction. Therefore, the profile of the terpene fraction present in the Cannabis extract is significantly different from the profile of the terpene fraction that exists in Nature.

A variety of terpenes and terpenoids have also been identified in Cannabis extracts, including monoterpenes, monoterpenoids, sesquiterpenes and sesquiterpenoids. For example, the following terpenes and terpenoids have been identified in Cannabis extracts: Alloaromadendrene, allyl hexanoate, benzaldehyde, (Z)-a-cis-bergamotene, (Z)-a-trans-bergamotene, ß-bisabolol, epi-a-bisabolol, ß-bisabolene, borneol (camphol), cis-y-bisabolene, bomeol acetate (bomyl acetate), α-cadinene, camphene, camphor, cis-carveol, caryophyllene (ß-caryophyllene), α-humulene (α-caryophyllene), γ-cadinene, Δ-3-carene, caryophyllene oxide, 1,8-cineole, citral A, citral B, cinnameldehyde, α-copaene (aglaiene), γ-curcumene, ß-cymene, ß-elemene, γ-elemene, ethyl decdienoate, ethyl maltol, ethyl propionate, ethylvanillin, eucalyptol, α-eudesmol, ß-eudesmol, γ-eudesmol, eugenol, cis-ß-famesene ((Z)-ß-farnesene), trans-α-farnesene, trans-ß-famesene, trans-γ-bisabolene, fenchone, fenchol (norbomanol, ß-fenchol), geraniol, α-guaiene, guaiol, methyl anthranilate, methyl salicylate, 2-methyl-4-heptanone, 3-methyl-4-heptanone, hexyl acetate, ipsdienol, isoamyl acetate, lemenol, limonene, d-limonene (limonene), linolool (linalyl alcohol, ß-linolool), α-longipinene, menthol, γ-muurolene, myrcene (ß-myrcene), nerolidol, trans-nerolidol, nerol, ß-ocimene (cis-ocimene), octyl acetate, α-phellandrene, phytol, α-pinene (2-pinene), ß-pinene, pulegone, sabinene, cis-sabinene hydrate (cis-thujanol), ß-selinene, α-selinene, γ-terpinene, terpinolene (isoterpine), terpineol (α-terpineol), terpineol-4-ol, α-terpinene (terpilene), α-thujene (origanene), vanillin, viridiflorene (ledene), and α-ylange.

It is believed that the presence of the particular terpenes/terpenoids in the terpene fraction is associated with beneficial effects of the pharmaceutical composition in use.

The terpene fraction may comprise one or more of beta-myrcene, linalool, nerolidol, limonene, alpha-bisabolol, camphene, delta-s-carene, beta-caryophyllene, caryophyllene oxide, p-cymene, geraniol, humulene, ocimene, pinene, and alpha-terpinene.

Preferably, the extract comprises beta-myrcene. It is believed that beta-myrcene may enhance the bioavailability of the cannabinoids present in the extract. Beta-myrcene may be present in an amount of from 0% to about 40% by weight of the extract. In some embodiments, beta-myrcene is present in an amount of about 0-40% by weight of the terpene fraction, for example, from 0.001% to about 25%, 5.1% to 29% or about 5.5% to about 25% of the terpene fraction.

The terpene fraction may further comprise one or more of linalool, nerolidol and limonene.

When present, the limonene may be present in an amount of at least about 5.4% by weight of the terpene fraction, for example, from about 5.5% to about 50% or about 5.5% to about 20% by weight of the terpene fraction. Limonene is a cyclic monoterpene having the molecular formula C₁₀H₁₆. There are a number of different naturally occurring isomers; however, the most common form is the dextrorotatory isomer, namely D-limonene.

Linalool is a terpenoid that is found in many flower and spice plants having the molecular formula C₁₀H₁₈. It is believed that when linalool is present in a Cannabis extract, that is may provide a sedative effect. In some embodiments, linalool may be present in an amount of at least 0.05% by weight of the terpene fraction. In some preferred embodiments, linalool is present in an amount of greater than 4.5% by weight (e.g. at least 5% by weight of the terpene fraction). In other embodiments, linalool is present in an amount of from 0.05% to 25% by weight of the terpene fraction, for example, from 0.1% to 20% or 5% to 20% by weight of the terpene fraction.

Nerolidol is a sesquiterpenoid having the molecular formula of C₁₅H₂₆O. It exists in Nature in two isomeric forms, namely nerolidol 1 and nerolidol 2, which differ in the geometry around a central olefin, i.e. either cis or trans isomers. The extract may comprise nerolidol (i.e. nerolidol 1 and nerolidol 2) in an amount of at least 0.001% by weight of the terpene fraction, for example, from 0.01% to 20% by weight of the terpene fraction. Nerolidol 2 may be present in a greater amount relative to nerolidol 1. In some embodiments, nerolidol 1 may be absent (or present in an amount below the limit of detection). In some embodiments, nerolidol 2 may be absent (or present in an amount below the limit of detection). In some embodiments, nerolidol 1 and nerolidol 2 are absent (or present in an amount below the limit of detection). Nerolidol 1 may be present in the extract in an amount of at least about 0.001% by weight of the terpene fraction, for example, from 0.001% to 20% or 0.001 to 15% by weight of the terpene fraction. Nerolidol 2 may be present in the extract in an amount of at least about 0.001% by weight of the terpene fraction, for example, from 0.001% to 30% or 1% to 25% by weight of the terpene fraction.

The Cannabis extract may also comprise a pinene (e.g. alpha-pinene and/or beta-pinene). Pinene is a bicyclic monoterpene having the molecular formula C₁₀H₁₆. Pinene is found in Nature in two isomeric forms: alpha-pinene and beta-pinene. The extract may comprise pinene (i.e. alpha-pinene and beta-pinene) in an amount of at least 5% by weight of the terpene fraction, for example, at least 6%, 7%, 8%, 9% or 10% by weight of the terpene fraction. Typically, alpha-pinene may be present in an amount greater than the amount of beta-pinene. The ratio of beta-pinene to alpha-pinene may be about 4:1. Alpha-pinene may be present in the extract in an amount of at least about 0.001% by weight of the terpene fraction, for example, from 0.001% to 30%, 0.001% to 20% or 5% to 20% by weight of the terpene fraction. Beta-pinene may be present in the extract in an amount of at least about 0.001% by weight of the terpene fraction, for example, 0.001% to 25%, 1% to 25% or 1% to 10% by weight of the terpene fraction.

The terpene fraction may also comprise beta-caryophyllene. Beta-caryophyllene may be present in an amount of at least 0.001% by weight of the terpene fraction, for example, from 0.001% to 20% or 0.001% to 10% of the terpene fraction.

The terpene fraction may also comprise caryophyllene oxide. Caryophyllene oxide may be present in an amount of at least 0.001% by weight of the terpene fraction, for example, from 0.001% to 50%, 5% to 40%, 10% to 40% or 20% to 40% by weight of the terpene fraction.

In some embodiments, the extract further comprises humulene. It is believed that that humulene may enhance the sedative properties of the extract. Humulene is also sometimes called alpha-caryophyllene.

The Cannabis extract may also include ocimene. Ocimene may be present in an amount of at least 0.001% by weight of the terpene fraction, for example, from 0.001% to 20% or 0.001% to 5% by weight of the terpene fraction.

In some embodiments, specific terpenes or terpenoids may be absent, or present in non-detectable amounts (e.g. less than 0.001% by weight of the analyte). In some embodiments, one or more of the following terpenes or terpenoids are absent, or present in non-detectable amounts: alpha-bisabolol, delta-s-carene, geraniol, guaiol, isopulegol, limonene, nerolidol 1, nerolidol 2, gamma-terpinene, and terpinolene.

The cannabinoid fraction and the terpene fraction for two exemplary pharmaceutical compositions are set out in the following Tables 1 and 2. Amounts of cannabinoids are reported as determined by high-performance liquid chromatography (HPLC) and amounts of terpenes are reported as determined by gas chromatography (GC). It will be appreciated that, as the Cannabis extract is derived from Nature, the amount of each component may vary in some cases by +/−10%, +/−25% or +/−50%. The ranges of amounts corresponding to each of these limits to account for the potential variation in the composition are also shown in Table 1 and 2.

TABLE 1
THC-rich pharmaceutical composition

| | Amount (wt % | | | |
|---|---|---|---|---|
| | of | | | |
| Compound | composition) | +/−10% | +/−25% | +/−50% |
| THCA | 0.000 | — | — | — |
| THC | 0.424 | 0.3816-0.4664 | 0.318-0.53 | 0.212-0.636 |
| THCV | 0.000 | — | — | — |
| CBD | 0.000 | — | — | — |
| CBDA | 0.000 | — | — | — |
| CBG | 0.064 | 0.0576-0.0704 | 0.048-0.08 | 0.032-0.096 |
| CBN | 0.000 | — | — | — |
| CBC | 0.000 | — | — | — |
| Cannabinoid | 0.488 | 0.4392-0.5368 | 0.366-0.61 | 0.244-0.732 |
| fraction | | | | |
| alpha-bisabolol | 0.000 | — | — | — |
| camphene | 0.004 | 0.0036-0.0044 | 0.003-0.005 | 0.002-0.006 |
| delta-s-carene | 0.001 | 0.0009-0.0011 | 0.00075-0.00125 | 0.0005-0.0015 |
| beta-caryophyllene | 0.003 | 0.0027-0.0033 | 0.00225-0.00375 | 0.0015-0.0045 |
| caryophyllene oxide | 0.031 | 0.0279-0.0341 | 0.02325-0.03875 | 0.0155-0.0465 |
| p-cymene | 0.009 | 0.0081-0.0099 | 0.00675-0.01125 | 0.0045-0.0135 |
| geraniol | 0.000 | — | — | — |
| guaiol | 0.000 | — | — | — |
| alpha-humulene | 0.001 | 0.0009-0.0011 | 0.00075-0.00125 | 0.0005-0.0015 |
| isopulegol | 0.000 | — | — | — |
| D-limonene | 0.000 | — | — | — |
| linalool | 0.013 | 0.0117-0.0143 | 0.00975-0.01625 | 0.0065-0.0195 |
| beta-myrcene | 0.005 | 0.0045-0.0055 | 0.00375-0.00625 | 0.0025-0.0075 |
| nerolidol 1 | 0.000 | — | — | — |
| nerolidol 2 | 0.000 | — | — | — |
| ocimene | 0.003 | 0.0027-0.0033 | 0.00225-0.00375 | 0.0015-0.0045 |
| alpha-pinene | 0.015 | 0.0135-0.0165 | 0.01125-0.01875 | 0.0075-0.0225 |
| beta-pinene | 0.004 | 0.0036-0.0044 | 0.003-0.005 | 0.002-0.006 |
| alpha-terpinene | 0.001 | 0.0009-0.0011 | 0.00075-0.00125 | 0.0005-0.0015 |
| gamma-terpinene | 0.001 | 0.0009-0.0011 | 0.00075-0.00125 | 0.0005-0.0015 |
| terpinolene | 0.000 | — | — | — |
| Terpene fraction | 0.092 | 0.0828-0.1012 | 0.069-0.115 | 0.046-0.138 |
| Total Cannabis | 0.537 | 0.4833-0.5907 | 0.40275-0.67125 | 0.2685-0.8055 |
| extract in | | | | |
| pharmaceutical | | | | |
| composition | | | | |

Notes:
Amounts shown as 0 wt % either indicate that the compound was not detected or present in an amount below the detection limit (e.g. less than 0.005 mg/gram)

TABLE 2
CBD-rich pharmaceutical composition

| | Amount | | | |
|---|---|---|---|---|
| | (wt % of | | | |
| Compound | composition) | +/−10% | +/−25% | +/−50% |
| THCA | 0.005 | 0.0045-0.0055 | 0.00375-0.00625 | 0.0025-0.0075 |
| THC | 0.697 | 0.6273-0.7667 | 0.52275-0.87125 | 0.3485-1.0455 |
| THCV | 0.000 | — | — | — |
| CBD | 0.006 | 0.0054-0.0066 | 0.0045-0.0075 | 0.003-0.009 |
| CBDA | 0.000 | — | — | — |
| CBG | 0.013 | 0.0117-0.0143 | 0.00975-0.01625 | 0.0065-0.0195 |
| CBN | 0.008 | 0.0072-0.0088 | 0.006-0.01 | 0.004-0.012 |
| CBC | 0.011 | 0.0099-0.0121 | 0.00825-0.01375 | 0.0055-0.0165 |
| Cannabinoid | 0.729 | 0.6561-0.8019 | 0.54675-0.91125 | 0.3645-1.0935 |
| fraction | | | | |
| alpha-bisabolol | 0.002 | 0.0018-0.0022 | 0.0015-0.0025 | 0.001-0.003 |
| camphene | 0.006 | 0.0054-0.0066 | 0.0045-0.0075 | 0.003-0.009 |
| delta-s-carene | 0.000 | — | — | — |
| beta-caryophyllene | 0.004 | 0.0036-0.0044 | 0.003-0.005 | 0.002-0.006 |
| caryophyllene | 0.060 | 0.054-0.066 | 0.045-0.075 | 0.03-0.09 |
| oxide | | | | |
| p-cymene | 0.027 | 0.0243-0.0297 | 0.02025-0.03375 | 0.0135-0.0405 |
| geraniol | 0.011 | 0.0099-0.0121 | 0.00825-0.01375 | 0.0055-0.0165 |
| guaiol | 0.000 | — | — | — |
| alpha-humulene | 0.032 | 0.0288-0.0352 | 0.024-0.04 | 0.016-0.048 |
| isopulegol | 0.000 | — | — | — |
| D-limonene | 0.011 | 0.0099-0.0121 | 0.00825-0.01375 | 0.0055-0.0165 |
| linalool | 0.025 | 0.0225-0.0275 | 0.01875-0.03125 | 0.0125-0.0375 |
| beta-myrcene | 0.014 | 0.0126-0.0154 | 0.0105-0.0175 | 0.007-0.021 |
| nerolidol 1 | 0.000 | — | — | — |
| nerolidol 2 | 0.060 | 0.054-0.066 | 0.045-0.075 | 0.03-0.09 |
| ocimene | 0.005 | 0.0045-0.0055 | 0.00375-0.00625 | 0.0025-0.0075 |
| alpha-pinene | 0.043 | 0.0387-0.0473 | 0.03225-0.05375 | 0.0215-0.0645 |
| beta-pinene | 0.011 | 0.0099-0.0121 | 0.00825-0.01375 | 0.0055-0.0165 |
| alpha-terpinene | 0.015 | 0.0135-0.0165 | 0.01125-0.01875 | 0.0075-0.0225 |
| gamma-terpinene | 0.000 | — | — | — |
| terpinolene | 0.000 | — | — | — |
| Terpene fraction | 0.272 | 0.2448-0.2992 | 0.204-0.34 | 0.136-0.408 |
| Total Cannabis | 0.893 | 0.8037-0.9823 | 0.66975-1.11625 | 0.4465-1.3395 |
| extract in | | | | |
| pharmaceutical | | | | |
| composition | | | | |

Notes:
Amounts shown as 0 either indicate that the compound was not detected or present in an amount below the detection limit (e.g. less than 0.005 mg/gram)

The pharmaceutical composition may further comprise one or more pharmaceutically acceptable carriers, diluents, adjuvants, excipients or any combination thereof.

The carrier, diluent, adjuvant and/or excipient are “pharmaceutically acceptable” meaning that they are compatible with the other ingredients of the composition and are not deleterious to a subject upon or following administration. The pharmaceutical compositions may be formulated, for example, by employing conventional solid or liquid vehicles or diluents, as well as pharmaceutical additives of a type appropriate to the mode of desired administration (for example, excipients, binders, preservatives, stabilisers, etc.) according to techniques such as those well known in the art of pharmaceutical formulation (See, for example, Remington: The Science and Practice of Pharmacy, 21st Ed., 2005, Lippincott Williams & Wilkins). The pharmaceutically acceptable carrier may be any carrier included in the United States Pharmacopeia/National Formulary (USP/NF), the British Pharmacopoeia (BP), the European Pharmacopoeia (EP), or the Japanese Pharmacopoeia (JP). In some embodiments, the carrier, diluent, adjuvant and/or excipient may be non-natural (e.g. synthetically produced).

The pharmaceutical composition includes those suitable for topical administration, typically via direct application to the skin (e.g. the epidermal layer of the skin) of a patient.

The Cannabis extract, together with a conventional adjuvant, carrier, excipient or diluent, may thus be placed into the form of pharmaceutical compositions and unit dosages thereof, and in such form may be employed as a solid, such as rubs or powders to be dispersed in a liquid carrier prior to administration, or as a liquid, such as solutions, suspensions, emulsions, or oils. Liquid compositions are preferred.

Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For preparing pharmaceutical compositions from the Cannabis extract described herein, pharmaceutically acceptable carriers can be either solid or liquid. Solid form preparations include powders, cachets, and dispensable granules. A solid carrier can be one or more substances which may also act as diluents, lubricants, suspending agents, binders, preservatives, or an encapsulating material.

Suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a dosage form in which the active component, with or without carriers, is surrounded by a carrier, which is thus in association with it.

Liquid form compositions include sterile solutions, suspensions, emulsions, syrups, oils and elixirs. The Cannabis extract can be suspended in a pharmaceutically acceptable carrier, such as sterile water, sterile organic solvent or a mixture of both.

Other liquid form preparations include those prepared by combining the Cannabis extract with one or more naturally derived oils (e.g. an essential oil) or waxes. An “essential oil” is an oil derived by extraction (e.g. steam extraction, or contacting the plant material with an extractant) or pressing, which contains primarily hydrophobic, and generally fragrant, components of the plant material. Suitable naturally derived oils and waxes include any of those mentioned for the topical delivery system described above, including: Bergamot essential oil, Cedarwood essential oil, Chamomile essential oil, Clary sage essential oil, Cypress essential oil, Eucalyptus essential oil, Fennel essential oil, Frankincense essential oil, Geranium essential oil, Hyssop essential oil, Jasmine essential oil, Juniper essential oil, Lavender essential oil, Lemon essential oil, Lemongrass essential oil, Marjoram essential oil, Melaleuca essential oil, Myrrh essential oil, Myrtle essential oil, Neem essential oil, Orange essential oil, Oregano essential oil, Palma rosa essential oil, Patchouli essential oil, Peppermint essential oil, Rose essential oil, Rosemary essential oil, Rosewood essential oil, Sage essential oil, Sandalwood essential oil, Tangerine essential oil, Tea tree essential oil, Thyme essential oil, Ylang ylang essential oil, Sesame oil, Olive oil, Arnica essential oil, Lavender Spike essential oil, Cinnamon Leaf essential oil, Rosemary Cineole essential oil, Coconut oil, Bees wax and Hemp oil.

The amount of active ingredient in therapeutically useful compositions should be sufficient that a suitable dosage will be obtained.

Various other materials may be present to modify the physical form of the dosage unit. For instance, the composition may contain the Cannabis extract together with a preservative (e.g. methyl and propylparabens), and/or a dye. Of course, any material used in preparing any dosage unit form should be pharmaceutically acceptable and substantially non-toxic in the amounts employed. In addition, the active compound(s) may be incorporated into sustained-release preparations and formulations.

Pharmaceutically acceptable carriers and/or diluents include any and all solvents, dispersion media, coatings, antibacterial and antifungal agents, isotonic and absorption delaying agents and the like.

Also included are solid form preparations that are intended to be converted, shortly before use, to liquid form preparations. Such liquid forms include solutions, suspensions, and emulsions. These preparations may contain, in addition to the active component, colorants, stabilisers, buffers, dispersants, thickeners, solubilising agents, and the like.

For topical administration to the epidermis the active ingredients may be formulated as ointments, creams, oils or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

In the case of a spray, this may be achieved for example by means of a metering atomising spray pump. To improve nasal delivery and retention the compounds according to the invention may be encapsulated with cyclodextrins, or formulated with other agents expected to enhance delivery and retention in the nasal mucosa.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, for example in vials or ampoules. Also, the unit dosage form can be the dosage form itself, or it can be the appropriate number of these dosage forms in packaged form.

Also described herein are compositions absent a carrier where the compositions are in unit dosage form. Accordingly, also provided is a medicament comprising the Cannabis extract.

In some embodiments, the pharmaceutical composition further comprises an active agent other than the Cannabis extract. Any suitable active agent may be used provided that the activity of the active agent and/or the Cannabis extract is not diminished when combined.

### Methods of Treatment

In another aspect, also provided is a method for treating a skin disorder. The method comprising administering to a patient in need thereof an effective amount of the pharmaceutical composition or topical pharmaceutical composition described herein.

The pharmaceutical compositions may be used to treat a skin disorder. As used herein, reference to “skin disorder” includes diseases and disorders of the skin. Diseases or disorders of the skin include: acne, alopecia areata, basal cell carcinoma, Bowen's disease, congenital erythropoietic porphyria, contact dermatitis, Darier's disease, dystrophic epidermolysis bullosa, eczema (atopic eczema), epidermolysis bullosa simplex, erythropoietic protoporphyria, fungal infections of nails, Hailey-Hailey disease, herpes simplex, hidradenitis suppurativa, hirsutism, hyperhidrosis, ichthyosis, impetigo, keloids, keratosis pilaris, lichen planus, lichen sclerosus, melanoma, melasma, pemphigus vulgaris, plantar warts (verrucas), pityriasis lichenoides, polymorphic light eruption, psoriasis, pyoderma gangrenosum, rosacea, scabies, shingles, squamous cell carcinoma, Sweet's syndrome, vitiligo, or a combination thereof.

By “effective amount” it is meant an amount sufficient that when administered to the patient an amount of the drug is provided to achieve an effect. In the case of a therapeutic method, this effect may be the treatment of the skin disorder. Therefore, the “effective amount” may be a “therapeutically effective amount”. By “therapeutically effective amount” it is meant an amount sufficient that when administered to the patient an amount of drug is provided to treat the disease ora symptom of the disease.

As used herein, the terms “treating”, “treatment”, “treat” and the like mean affecting a subject, tissue or cell to obtain a desired pharmacological and/or physiological effect. The effect may be prophylactic in terms of completely or partially preventing, or reducing the severity of, a disease or associated symptom, and/or may be therapeutic in terms of a partial or complete cure of a disease. A reference to “treating” a skin disorder therefore encompasses: (a) arresting spreading of the skin disease; (b) reducing the area of skin affected by the skin disorder; (c) relieving or ameliorating the effects of the skin disorder, e.g. reducing visible signs of the disorder, or reducing irritation caused by the skin disorder; or (d) preventing the skin disorder from occurring in a subject predisposed to, or at risk of, the skin disorder, so that the skin disorder does not develop or occur in the subject, or presents in a less severe form.

The method may also comprise administering an active agent other than the Cannabis extract. This active agent may be administered simultaneously or consecutively with the Cannabis extract. By consecutively it is meant that each of the Cannabis extract and the other active agent are administered separately and may be at different times. Typically, when the Cannabis extract and the other active agent are administered consecutively they are administered within 24 hours, or within 12, 8, 6, 5, 4, 3, 2, or 1 hour(s) of each other. The Cannabis extract may be administered before or after the other active agent. Further, the route of administration for the Cannabis extract and the other active agent may be the same or different.

In another aspect, also provided is the use of the Cannabis extract in the preparation of a medicament for the treatment of the skin disorder.

Also provided is a kit comprising in separate parts:
(a) an effective amount of the Cannabis extract; and
(b) a pharmaceutically acceptable carrier, diluent, adjuvant, excipient or a combination thereof.

In some embodiments, the kit further comprises a part comprising (b′) an effective amount of an active agent other than the Cannabis extract. Part (b′) may be included in the kit, in addition to parts (a) and (b), or in place of part (b).

In another aspect, there is provided the pharmaceutical composition for treating the skin disorder. The pharmaceutical composition may be any of the pharmaceutical compositions described above, comprising any above-described combination of components, provided that it comprises the Cannabis extract. The skin disorder may also be any of those described above.

### EXAMPLES

The invention will be further described by way of non-limiting examples. It will be understood to persons skilled in the art of the invention that many modifications may be made without departing from the spirit and scope of the invention.

### Example 1—Cannabis Extracts

The following Cannabis extracts are described:

AZ9—combination of extracts of multiple Cannabis plants.

AZ10—extract of Ogre King plant.

| | AZ9 | AZ10 |
|---|---|---|
| Component | wt %³ | wt %³ |
| Cannabinoids¹ | | |
| THCA | ND | 0.005 |
| THC | 0.424 | 0.697 |
| THCV | ND | 0.000⁴ |
| CBD | ND | 0.006 |
| CBDA | ND | ND |
| CBG | 0.064 | 0.013 |
| CBN | ND | 0.008 |
| CBC | ND | 0.011 |
| Cannabinoid fraction | 0.488 | 0.729 |
| Terpenes² | | |
| alpha-bisabolol | ND | 0.002 |
| camphene | 0.004 | 0.006 |
| delta-s-carene | 0.001 | 0.000 |
| beta-caryophyllene | 0.003 | 0.004 |
| caryophyllene oxide | 0.031 | 0.060 |
| p-cymene | 0.009 | 0.027 |
| geraniol | ND | 0.011 |
| guaiol | ND | ND |
| alpha-humulene | 0.001 | 0.032 |
| isopulegol | ND | 0.000 |
| D-limonene | ND | 0.011 |
| linalool | 0.013 | 0.025 |
| beta-myrcene | 0.005 | 0.014 |
| nerolidol 1 | ND | ND |
| nerolidol 2 | ND | 0.060 |
| ocimene | 0.003 | 0.005 |
| alpha-pinene | 0.015 | 0.043 |
| beta-pinene | 0.004 | 0.011 |
| alpha-terpinene | 0.001 | 0.015 |
| gamma-terpinene | 0.001 | 0.000 |
| terpinolene | ND | 0.000 |
| total terpenes | 0.092 | 0.272 |
| Total | 0.537 | 0.893 |

Notes:
¹Cannabinoids were detected using HPLC analysis, an amount reported as 0 wt % indicates that the compound was either not detected, or present in an amount below the detection limit of the HPLC;
²Terpenes were detected using GC analysis, an amount reported as 0 wt % indicates that the compound was either not detected, or present in an amount below the detection limit of the GC;
³In order to allow for Natural variation, amount within +/−10%, +/−25% or +/−50% of the reported values;
⁴detected at 0.004 mg/g of analyte.

### Example 2—Formulation of Cannabis Extract into Topical Formulation

The Cannabis extract of Example 1 (AZ9 or AZ10) may be formulated into a topical formulation. The extract may be diluted to form a 50-80% solution of the extract. The extract or the 60-80% diluted extract may then be combined with about 16 litres of coconut oil and about 3 kilograms of bees wax, and about 1-100 millilitres each of the following components: Arnica essential oil, Lavender essential oil, Lavender Spike essential oil, Frankincense essential oil, Lemongrass essential oil, Cinnamon Leaf essential oil, Rosemary Cineole essential oil, Rosemary essential oil, Bergamot essential oil, Myrrh essential oil, and Sage essential oil. The topical formulation thus prepared may be used in the methods of treating a skin disorder described herein.

Unless the context requires otherwise, all percentages referred to herein are percentages by weight of the pharmaceutical composition.

The term “about”, when used to describe a value, preferably means an amount within ±10% of that value.

The terms “a”, “an”, “and” and/or “the” and similar referents in the context of describing the invention and the claims which follow are to be construed to cover both the singular and the plural, unless otherwise indicated herein or clearly contradicted by context.

It is to be understood that, if any prior art publication is referred to herein, such reference does not constitute an admission that the publication forms a part of the common general knowledge in the art, in Australia or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A pharmaceutical composition comprising a Cannabis extract and optionally one or more pharmaceutically acceptable carriers, diluents, adjuvants, excipients or any combination thereof, the Cannabis extract comprising at least 75% by weight of a main cannabinoid; wherein the pharmaceutical composition comprises a terpene fraction which fraction comprises limonene in an amount of at least about 5.4% by weight of the terpene fraction.

2. The pharmaceutical composition according to claim 1, where in the main cannabinoid is Δ⁹-Tetrahydrocannabinol (THC) or cannabidiol (CBD).

3. The pharmaceutical composition according to claim 1, wherein the Cannabis extract further comprises one or more secondary cannabinoids.

4. The pharmaceutical composition according to claim 3, wherein the one or more secondary cannabinoids are selected from Cannabinodiol (CBN), Cannabichromanone (CBC), Δ⁹-Tetrahydrocannabinolic acid (THCA) and Cannabigerol (CBG).

5. The pharmaceutical composition according to claim 4, wherein the terpene fraction is in an amount of at least 0.05% by weight.

6. The pharmaceutical composition according to claim 1, wherein Cannabis extract comprises one or more of beta-myrcene, linalool, nerolidol, alpha-bisabolol, camphene, delta-s-carene, beta-caryophyllene, caryophyllene oxide, p-cymene, geraniol, humulene, ocimene, pinene, and alpha-terpinene.

7. The pharmaceutical composition according to claim 1, comprising linalool in an amount of at least 4.5% by weight of the terpene fraction.

8. The pharmaceutical composition according to claim 1, for treating a skin disorder.

9. A topical pharmaceutical composition comprising an effective amount of a Cannabis extract comprising at least 75% by weight of a main cannabinoid and a terpene fraction which fraction comprises limonene in an amount of at least about 5.4% by weight of the terpene fraction and a topical delivery system.

10. (canceled)

11. The topical pharmaceutical composition according to claim 9, wherein the topical delivery system comprises two or more of Bergamot essential oil, Cedarwood essential oil, Chamomile essential oil, Clary sage essential oil, Cypress essential oil, Eucalyptus essential oil, Fennel essential oil, Frankincense essential oil, Geranium essential oil, Hyssop essential oil, Jasmine essential oil, Juniper essential oil, Lavender essential oil, Lemon essential oil, Lemongrass essential oil, Marjoram essential oil, Melaleuca essential oil, Myrrh essential oil, Myrtle essential oil, Neem essential oil, Orange essential oil, Oregano essential oil, Palma rosa essential oil, Patchouli essential oil, Peppermint essential oil, Rose essential oil, Rosemary essential oil, Rosewood essential oil, Sage essential oil, Sandalwood essential oil, Tangerine essential oil, Tea tree essential oil, Thyme essential oil, Ylang ylang essential oil, Sesame oil, Olive oil, Arnica essential oil, Lavender Spike essential oil, Cinnamon Leaf essential oil, Rosemary Cineole essential oil, Coconut oil, Bees wax and Hemp oil.

12. The topical pharmaceutical composition according to claim 9, wherein the topical delivery system comprises two or more of Sesame oil, Olive oil, Arnica essential oil, Lavender essential oil, Lavender Spike essential oil, Frankincense essential oil, Lemongrass essential oil, Cinnamon Leaf essential oil, Rosemary Cineole essential oil, Rosemary essential oil, Bergamot essential oil, Myrrh essential oil, Sage essential oil, Coconut oil, Bees wax and Hemp oil.

13. (canceled)

14. The topical pharmaceutical composition according to claim 9, for treating a skin disorder.

15. (canceled)

16. A method for treating a skin disorder, comprising administering to a patient in need thereof an effective amount of a composition according to claim 1.

17-18. (canceled)

19. The topical pharmaceutical composition according to claim 9, wherein the main cannabinoid is Δ⁹-Tetrahydrocannabinol (THC) or cannabidiol (CBD).

20. The topical pharmaceutical composition according to claim 9, wherein the Cannabis extract further comprises one or more secondary cannabinoids.

21. The topical pharmaceutical composition according to claim 11, wherein the one or more secondary cannabinoids are selected from Cannabinodiol (CBN), Cannabichromanone (CBC), Δ⁹-Tetrahydrocannabinolic acid (THCA) and Cannabigerol (CBG).

22. The topical pharmaceutical composition according to claim 9, wherein the terpene fraction is in an amount of at least 0.05% by weight.

23. The topical pharmaceutical composition according to claim 9, wherein Cannabis extract comprises one or more of beta-myrcene, linalool, nerolidol, alpha-bisabolol, camphene, delta-s-carene, beta-caryophyllene, caryophyllene oxide, p-cymene, geraniol, humulene, ocimene, pinene, and alpha-terpinene.

24. The topical pharmaceutical composition according to claim 9, comprising linalool in an amount of at least 4.5% by weight of the terpene fraction.
